(19) United States
(12) Patent Application Publication
(10) Pub. No.: US 2006/0195069 A1
(43) Pub. Date: Aug. 31, 2006

(54) Percutaneous diagnostic and therapeutic hematoma drain

(76) Inventors: John C. Opie, Scottsdale, AZ (US); Thomas Izdebski, Phoenix, AZ (US)

Correspondence Address: Squire, Sanders & Dempsey L.L.P., Two Renaissance Squire, 40 North Central Avenue, Suite 2700, Phoenix, AZ, 85004-4498, US

(21) Appl. No.: 11/236,207

(22) Filed: Sep. 26, 2005

Related U.S. Application Data
(60) Provisional application No. 60/612,909, filed on Sep. 24, 2004.

(51) Int. Cl. A61M 27/00

(52) U.S. Cl. 604/541

(57) ABSTRACT

The invention provides an over-the-needle, percutaneously-placed hematoma drain designed to aid in preventing, detecting, and draining a concealed hemorrhage while the drain is in place. It is designed to provide an early warning that vascular hemostasis following vascular access device removal may not be adequate for patient discharge.

### RELATED APPLICATIONS

This application claims priority to U.S. Provisional Patent Application No. 60/612,909 filed on Sep. 24, 2004 which is hereby incorporated by reference herein. In addition, the following related utility and provisional applications are also incorporated by reference herein:

“Surgical Drains” by John Opie et al.—U.S. Patent Publication No. 2005/0004536, published Jan. 6, 2005 from U.S. patent application Ser. No. 10/863,009 filed on Jun. 7, 2004 which claims priority from Provisional Application No. 60/476,663 filed on Jun. 5, 2003. “Fluid Evacuator System” by John Opie—PCT Publication No. WO-05/072789, published Aug. 11, 2005 from PCT Application No. US05/03128 filed on Jan. 26, 2005 which claims priority from U.S. Provisional Application No. 60/539,138 filed on Jan. 26, 2004.

### FIELD OF THE INVENTION

The present invention relates to medical devices, and more particularly, to an over-the-needle, percutaneously-placed drain designed to aid in preventing, detecting and/or draining a concealed hemorrhage while the drain is in place.

### BACKGROUND OF THE INVENTION

Unilateral or bilateral femoral artery cannulation (i.e., the insertion of a cannula or tube into a hollow body organ) for vascular access is a very common approach used in cardiology, interventional radiology and endovascular surgery for intra-vascular procedures, such as coronary artery stenting, endoluminal aortic grafting, carotid or renal artery stenting, rotational athrectomy, laser catheterization and the like that may be used to reopen chronically occluded blood vessels. The already large volume of these procedures performed is expected to further increase, particularly as the use of covered stents is becoming increasingly popular.

In procedures involving the femoral artery, procedures typically begin by shaving, prepping and draping the groin area. Once a sterile field is obtained the proceduralist sticks the groin over a femoral artery that can be palpated. A needle, e.g., a Potts needle, Cook needle, etc., is depressed into the femoral artery until blood returns. At that point, a guide wire is passed up the needle and into the femoral artery and confirmed in an intra-arterial position by fluoroscopy. Heparin is now given to render the patient unable to clot. The needle is then removed and a sheath is passed over the guide wire and into the femoral/iliac artery. After the sheath is placed, secondary procedures then typically occur.

Existing sheaths range from small diameter 4Fr (⅓ mm=1 Fr), up to 20-27Fr large bore sheaths. Sheaths larger than 7Fr create a substantial hole in the femoral artery wall. The arterial penetration may be in the direct anterior wall or it may be entered from either side. There can be posterior wall penetration with the initial needle sticks. The penetration can inadvertently be high in the external iliac artery. These latter arterial punctures set up the potential for a hematoma (i.e., a localized swelling filled with blood resulting from a break in a blood vessel) due to sheath pull out pressure control problems. Once the procedure is completed the anti-coagulant effects may be reversed with protamine or it may be left to correct by nature. Once the PTT (a test used to evaluate blood clotting) has normalized, the sheath is pulled.

Currently, two techniques are used to limit blood extravasations (i.e., blood exuding from a blood vessel into surrounding tissues) into the inguinal and thigh tissues upon sheath removal at the end of the procedure. The most common is simple direct pressure over the probable puncture site in the artery by a hand or a mechanical device such as a “Fem-Stop.” Adjunct procedures may include “Per-Close” or “Angio-Seal.” These devices include a variety of collagen deposits or rudimentary mechanical suture systems. The main problem with the latter devices is that they are applied in a blind percutaneous (i.e., through the skin) manner and thus can and do fail. In certain situations, the site selected for pressure and or closure, entirely misses the femoral artery puncture site with adverse consequences for the patient. With some devices, ultrasound guidance is used with possible beneficial effect.

Failure under these circumstances results in a concealed hematoma, which may develop into a substantial false aneurysm (i.e., a pulsating, encapsulated hematoma communicating with a ruptured vessel) and require definitive vascular surgical repair. Many of these hematomas resolve without additional intervention, provided the femoral artery bleeding stops. However, this may take some hours. Such hematomas are associated with the development of anemia, marked bruising, and dramatic skin discolorization—often from knee to umbilicus and all points in between. The patient typically experiences pain for days if not weeks and has difficulty bending the hip joint. Not uncommonly, major problems develop and anterior thigh compartment compression syndrome occurs. This leads to pronounced patient distress due to a large or very large concealed hemorrhage. The distress can present as hypotension, giant groin hematomas, skin blistering and sloughing, groin sepsis and major skin loss secondary to tension related local ischemia. In extreme situations, such hematomas may result in leg amputation and rarely death.

Most of these large hematomas will require an urgent visit to the OR. Surgery includes hematoma evacuation and suturing of the bleeding artery puncture site. Not infrequently a blood transfusion is required for hemodynamic stability. The process is both exciting and dangerous for all concerned. Surgical dexterity and an anxious proceduralist become the order of the day.

Current technology offers “blind” techniques for stopping and preventing arterial leaks. However, such “blind” techniques can and do fail. As such, current technology does not provide desirable methods and devices for detection, prevention, and early correction of failures and hematomas resulting from vascular procedures.

### SUMMARY OF THE INVENTION

In view of the foregoing, the present invention provides a percutaneously-placed diagnostic and therapeutic hematoma drain. Preferably, the drain is used to detect and drain hemorrhages resulting from vascular procedures.

The percutaneously-placed body-fluid drain according to the invention includes a main body including a central lumen, the main body having a distal end and a proximal end, a distal tip at the distal end of the main body, the distal tip having a low-profile shape for aiding insertion into a patient, one or more side holes in the distal tip, the side holes being in fluid communication with the central lumen, and a connector attached to the proximal end of the main body, the connector for connecting the drain to an outflow tube such that the outflow tube is in fluid communication with the central lumen.

When in place, the connector of the drain is connected to an outflow tube which is connected to a vacuum reservoir. A vacuum is created in the system and any blood or body fluids that may be leaking in the patient are sucked into the drain, through the outflow tube, and into the reservoir. As any blood moving into the reservoir is visible, the drain effectively unmasks a concealed hemorrhage following a vascular access procedure. In addition to unmasking the hemorrhage, the drain provides a pathway for blood to be vented from the body, thus preventing a complications from occurring.

It is to be understood that the descriptions of this invention herein are exemplary and explanatory only and are not restrictive of the invention as claimed.

### BRIEF DESCRIPTION OF THE DRAWINGS

FIG. 1 is a plan view of the drain according to one embodiment of the invention.

FIG. 2 is a lateral view of the blunt needle and syringe assembly for use in drain insertion.

FIG. 3 is the assembled device ready for percutaneous insertion

FIG. 4 is a plan view showing the connecting tubing.

FIG. 5A is a perspective view of a container that may be used as part of an evacuator system for use with a drain.

FIG. 5B is a perspective view of a cover for the container of FIG. 5A.

### DESCRIPTION OF THE EMBODIMENTS

Reference will now be made in detail to the present exemplary embodiments of the invention, examples of which are illustrated in the accompanying drawings.

The hematoma drain according to the invention is an “early warning” percutaneously-placed drainage device designed as a diagnostic measure to alert the staff that hemostasis (i.e., the stoppage of bleeding or hemorrhage) might not be secure, and if a bleed does occur, it will evacuate blood before a hematoma forms and will help prevent greater complications. In addition, use of the hematoma drain will permit secondary procedures to occur earlier and will limit some occasional compression syndromes which might occur after the drain has been removed. The drain will be expected to become active only after the sheath is removed, however the drain may be active while the sheath is still in place. Typically, the period of use of the hematoma drain may vary between 1 and 3 hours before the sheath is pulled.

At the completion of any percutaneous vascular access procedure, the proceduralist should evaluate the potential to have inadequate hemostasis upon sheath removal. Larger sheaths, difficult access, and suspicion for external iliac artery access above the inguinal ligament are all indicators of potential increased hemostasis problems upon sheath removal. If groin bleeding does occur, but no drain is in the leg, then the groin will have to be managed as a concealed hemorrhage site as is done currently. In general, the larger the sheath and the more sticks to gain access, the more potential there is to get an access hematoma, and the prophylactic insertion of a hematoma drain according to the invention may avoid the complications.

The hematoma drain permits femoral artery bleeding to become visual and therefore unconcealed as it collects in the drain reservoir following removal of a vascular access device. Visual blood accumulating in the drain reservoir will be brought to the attention of the nursing and medical staff and permit steps to be taken to arrest the bleeding. This may include reapplying compression, altering the compression site, notifying the proceduralist, and checking the PTT/PT to ensure the patient is not anticoagulated. If these simple steps fail, earlier vascular surgery is indicated.

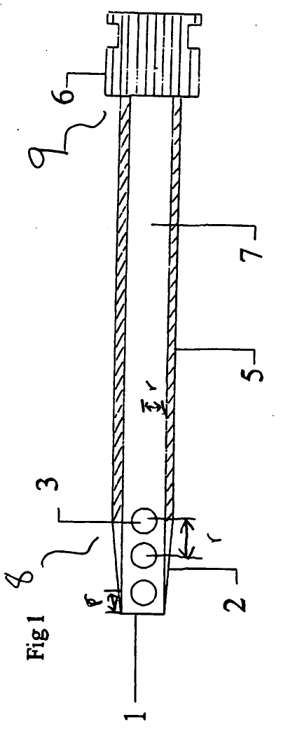

FIG. 1 shows one embodiment of a percutaneously-placed hematoma drain according to the invention. Drain 1 includes a distal tip 2, a main body 5, and a connector 6. Main body 5 has a distal end 8 and a proximal end 9. Preferably the main body is tubular in shape, however any shape suitable for percutaneous placement may be used. Running through the center of main body 5 is a lumen 7. The lumen provides fluid communication from the distal end through the main body to the proximal end. Preferably, the cross-section of the lumen has a cylindrical shape with a radius (r). The radius may be of any size that is suitable for placement over a needle for insertion into the placement, but generally need not be larger than 18-20 Fr.

As shown in FIG. 1, distal tip 2 is at distal end 8 of main body 5. As shown, distal tip 2 is chamfered. A chamfer is a flat surface made by cutting off the edge or corner. By chamfering the distal tip, a low-profile, streamlined shape is achieved. This allows for easier percutaneous insertion through an incision in the patient's skin. The chamfered distal end allows passage into the body without getting caught on the skin and subcutaneous tissues as it is depressed in to a position close to the artery before the sheath is removed and compression applied to the arterial puncture site. While chamfering is one method of creating the desired low-profile shape, any low-profile shape that aids in the insertion of the drain may be used.

One radius (r) (i.e., the radius of the central lumen) set back from the end of distal tip 2 are one or more opposing side holes 3. FIG. 1 depicts three side holes 3, however, more or fewer side holes may be employed. Side holes 3 are in fluid communication with central lumen 7 so that blood and body fluids are allowed to pass from the patient through the side holes and into the central lumen 7.

In addition to the side holes shown in FIG. 1, it also preferable to have a corresponding “opposing” side hole on the other side of the distal tip. As shown in FIG. 1, the distance between the centers of each side hole is the radius (r) of central lumen 7. In addition, the diameter of each of the side holes 3 is preferably the radius (r) of central lumen 7, however any diameter suitable for draining blood and body fluids may be used. In addition, FIG. 1 depicts side holes 3 as being arranged in a row. However, different arrangements and spacings of side holes 3 may be employed so long as they effectively draw blood and other body fluids when in place. Different side hole configurations and sizes are possible. These include round holes, oval shaped holes, and gill slits, either longitudinally or horizontally shaped. Smaller holes or larger holes are all possible and may be positioned in different configurations.

In addition to arrangements that include opposing side holes, additional side holes (or rows of side holes) may be positioned so that they are 90 degrees out of phase with the side holes depicted in FIG. 1. In this way, the distal tip 2 would include four side holes (or four rows of side holes) radially spaced about the tip. As such, this arrangement would facilitate near 360-degree fluid drainage. Additional side holes and side hole rows may be incorporated if found to provide improved drainage without sacrificing structural integrity.

Proximal to side holes 3 and distal tip 2 is main body 5. Preferably, main body 5 has no side holes for added structural integrity, however side holes may be employed in main body 5 so long as structural integrity is maintained. That is, main body 5 should remain in substantially tubular shape when inserted into the patient. In order to maintain this structural integrity, main body 5 is preferably made from a softer, semi deformable plastic that retains a tubular form while indwelling in the patient's body tissues. However, any material suitable for use as a percutaneously-placed drain may be employed so long as it maintains structural integrity when in use.

Proximal end 9 of main body 5 is bonded to a connector 6, which is preferably a standard female Luer Lock, however any connector for connecting drain 1 to a syringe or outflow tubing may be used. Preferably, connector 6 is made of plastic that is harder and more rigid than that employed by main body 5, however any suitable material for making the connector may be used.

Preferably, hematoma drain 1 is of sufficient length such that it protrudes outside the body such that it can be attached non-invasively to the patient's skin by adhesive tape. Such an attachment is similar to the attachment systems that are used for IVs. As such, hematoma drain 1 may be made in various lengths depending on the patient and procedure being performed. The length of distal tip 2 should be made long enough for sufficient drainage of hematomas. That is, the length of distal tip 2 is also dependent on the patient and procedure being performed. Typically, distal tip 2 is less than ⅓ the length of main body 5, however, longer distal tips may be employed in situations where the volume and size of potential hematomas may be large.

The potential for intercurrent clot formation within the drain 1 may be reduced with a non-thrombogenic (non-coagulating) coating. Such a coating would line central lumen 7 as well as side holes 3.

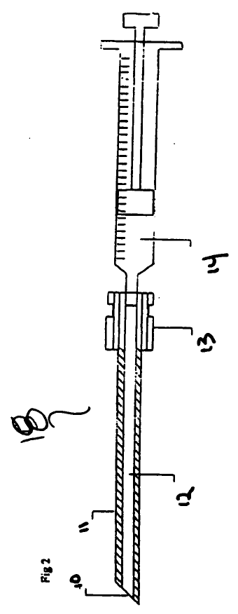

FIG. 2 is a view of a large bore needle used for insertion of drain 1. Needle 18 includes a blunt leading edge bevel 10, a main body 11 with a central lumen 12, and a connector 13, which is preferably a standard female Luer Lock. The connector 13 is designed to be attached to a syringe 14 for ease of insertion. The blunt leading edge bevel 10 is beveled to a point at approximately half the angle of a regular IV needle. Being noticeably blunter than an IV needle, it will be less likely to accidentally stick a vascular structure during drain placement and thereby promote additional bleeding. In addition, needle 18 may optionally be fitted with an obturator to prevent patient tissues for getting caught inside the needle when it is being inserted.

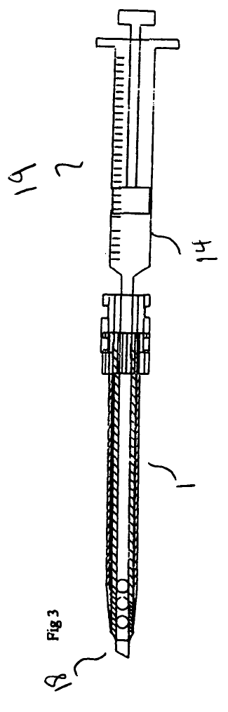

FIG. 3 is a view of the assembled device ready for percutaneous insertion. The assembled device 19 includes hematoma drain 1 fitting coaxially over needle 18. The needle is fitted to a syringe 14, which is preferably 5 or 10 cc syringe, via the proximal Luer Lock fittings. The proximal end of the needle terminates in a female Luer Lock. As such, it can fit a syringe to better facilitate placing the drain by providing a large gripping device and a better needle aiming facility.

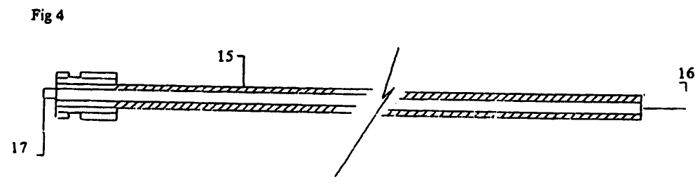

FIG. 4 is a view of the outflow tubing 15. Outflow tubing 15 is comprised of a highly flexible, kink resistant, outflow tube having a smooth exterior for sealing to surface tissue at a point of exit from a patient's body. The tubing has two ends. The proximal end 16 is an undistinguished end, which receives the spout of a portable vacuum reservoir such as the reservoir described in “Fluid Evacuator System” by John Opie—PCT Publication No. WO-05/072789, published Aug. 11, 2005 from PCT Application No. US05/03128 filed on Jan. 26, 2005 which claims priority from U.S. Provisional Application No. 60/539,138 filed on Jan. 26, 2004, which is incorporated by reference herein. The distal end of the outflow tubing is connected to a connector 17, which is preferably a male Luer lock end for connecting the tubing to the connector 6 of drain 1 when the drain is in place. The male configured distal end Luer lock 17 fits the female Luer Lock 6 of the drain 1 with which it locks into position and prevents air entering the vacuumed system.

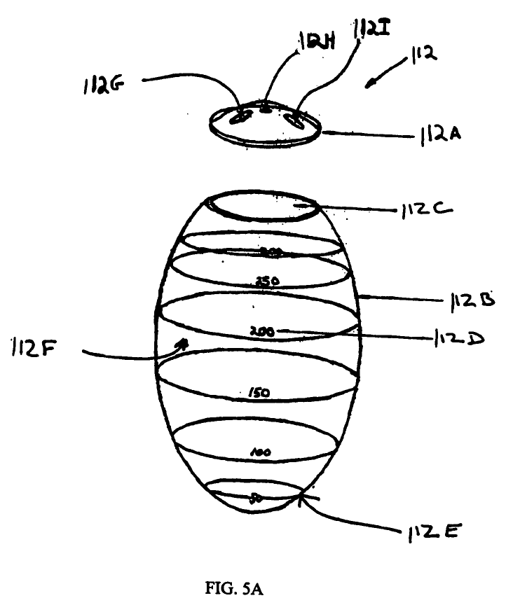

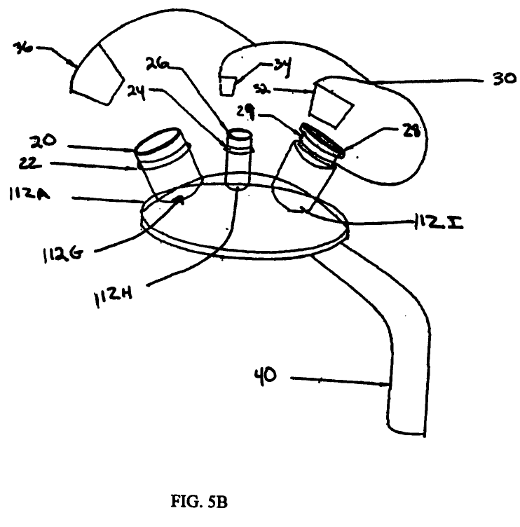

FIGS. 5A and 5B show a portable ellipsoid bulb or “grenade” style vacuum reservoir or fluid container 112. In one embodiment, fluid container 112 may include a container cover 112A and a container body 112B, which when assembled together defines an internal reservoir 112F. Container cover 112A and container body 112B may be assembled in any suitable manner prior to use, such as by heat sealing during manufacturing or by cover 112A being threadingly received on body 112B. Fluid container 112 includes one or more passages, such as passages 112G-1121, which permit passage of fluid, such as body fluid from a wound into reservoir 112F or from reservoir 112F to another container when fluid container 112 is being emptied. Fluid container 112 may also include markings 112D to indicate the quantity of fluid that may be collected in fluid container 112. For example, as shown in FIG. 5A, fluid container 112 may include markings 12D in 50 cubic-centimeter increments up to 300 cubic centimeters.

Vacuum is applied to draw body fluid from a wound site, via drain 1 and outflow tubing 15 into internal reservoir 112F. Any structure or device suitable for this purpose may be employed. Moreover, the vacuum may be created from expansion (or decompression) of fluid container 112 after it has been compressed, and/or from an external vacuum source (not shown) that provides vacuum to fluid container 112.

Fluid container 112 may comprise a single, integral part or multiple assembled parts. Fluid container 112 may be manufactured using any compressible and resilient material. As a result, fluid container 112, may be compressed and then allowed to expand to create vacuum suitable to draw, or assist in drawing, fluid from a wound into internal an reservoir 112F. When assembled, the uncompressed shape of fluid container 112 may generally resemble an ellipsoid, however, fluid container 112 may have any material property and any shape that is suitable for the purposes of an evacuator system.

When in use, fluid container 112 may lie on its side or be configured to stand upright, as shown in FIG. 5A. In the latter case, fluid container 112 may include a variation from the embodiment shown in FIG. 5A, namely having a lower section, such as section 112E, removed and sealed to create a flat surface to be supported. Such a variation may be completed during manufacture of fluid container 112. Any suitable surface may then be used to support fluid container 112 on an end in an upright manner. Fluid container 112 may also include any structure suitable for attaching fluid container 112 to any article, e.g., attaching fluid container 112 to an article such as clothing or bedding near a patient. A shown in FIG. 5B, a lanyard 40 and an attaching pin (not shown) may serve this purpose.

A variation of container cover 112A is shown in FIG. 5B. Extending from and in communication with passages 112G, 112H and 112I may be respective ports 20, 26 and 28. Port 26 may serve as a fluid input to reservoir 112F and may have a diameter corresponding to its respective passage 112H that accommodates a first type of outflow tube for a first drain. Port 20 may serve as a fluid input to reservoir 112F and may have a diameter corresponding to its respective passage 112G that accommodates a second type of conduit for a second drain.

Port 28 may serve as a fluid output from fluid container 112. Ports 20 and 26 may include respective retainers 22 and 24, such as chevrons, to retain respective outflow tubes (not shown), by pressing a respective outflow tube over the port. However, any structure for placing an outflow tube in fluid communication with internal reservoir 112F may be utilized.

Port 28 may include a neck 29 to receive a band 30 for retaining stoppers 36, 34 and 32 for their respective ports 20, 26 and 28. Any material may be used for lanyard 40, so that it may be reused to attach fluid container 112 to an article, such as an article of clothing or bedding near patient 14. Container cover 112A may also include one or more valves to prevent backflow through any input passages, such as passages 112G and 112H, back to a patient.

In operation, one of the fluid input ports 20 or 26 may be blocked with its respective stopper 36 or 34. The other input port 20 or 26 may be connected to a outflow tube that may be attached to a drain, e.g., drain 1, in a wound site or to be placed in a wound site. With the output port stopper 32 removed, fluid container 112 may be compressed to force fluid, e.g., air and/or body fluid, out of internal reservoir 112F through output port 28. The output port stopper 30 may then be reinserted. Fluid container 112 may then, due its resiliency, begin expanding to an uncompressed state, thus creating suction or vacuum via an outflow tube and a drain to remove body fluid from a wound site.

Now the insertion and use of the hematoma drain of the invention will be described.

At the completion of any percutaneous vascular access procedure, the proceduralist should evaluate the potential to have to inadequate hemostasis upon sheath removal. Larger sheaths, difficult access and suspicion for external iliac artery access above the inguinal ligament are all indicators of potential increased hemostasis problems upon sheath removal. If there is any doubt at all, it is recommended a the hematoma drain of the invention be used. In general, the larger the sheath and the more sticks to gain access, the more is the potential to get an access hematoma, and prophylactic hematoma drain insertion may eliminate the complications.

In the present state of the art, concealed hemorrhaging becomes a difficult diagnosis due to the loose areolar tissue in the groin, which can accept significant blood before serious signs become apparent. The hematoma drain according to the invention can be placed in the groin at the end of the procedure and before the sheath is removed by pressing the drain through an area of the skin that has received local anesthesia. The blunt needle can be felt touching the still indwelling sheath and be positioned to one side or immediately below and near by the sheath. The needle component of the drain is then removed and the plastic drain is then connected to a short tubing segment. The tubing is then connected to a collapsible ellipsoid portable vacuum reservoir.

The vascular access sheath is removed when the coagulation studies have normalized and compression applied in a routine manner.

An empty drain is removed before the patient is discharged according to the appropriate schedule. A drain full of blood should give rise to thought as to whether hemostasis is adequate. Such a blood filled reservoir may lead to additional compressive pressure after emptying the drain. At an appropriate time after the sheath has been pulled, if no additional bleeding has occurred, the drain can be pulled and the patient discharged with a greater level of comfort that a concealed hemorrhage has been avoided.

People skilled in the art of percutaneous vascular access will have little difficulty in placing the hematoma drain of the invention. Because the assembled system is of a larger diameter than most needle sticks, but typically not larger than 18-20Fr vascular access sheaths used, for example, in endoluminal aortic aneurysm repairs, most proceduralists who may complete these procedures will not be unduly concerned by the size, e.g., diameter, of the hematoma drain 1.

Once the endovascular procedure is finished, all wires, cannulae and deployment devices are removed. Heparin, if it is to be reversed, should be done with protamine at that time. On many occasions, it will be left to naturally correct.

While in the catheterization laboratory with only the sheath remaining, hematoma drain 1 should be brought into the sterile field. Before insertion, drain 1 should be moved up and down needle 18 to make sure it can be slid off the needle with ease. It may be necessary to wet the needle and central lumen 7 of drain 1 to make this disengagement movement slippery. Once that is done, a site within the local anesthetic area should be chosen either medial to (femoral vein) or lateral to (femoral nerve) or below the sheath (femoral artery). Below the sheath is probably the preferred site and drain 1 should be angled either laterally or medially as it is placed. Once the placement site is confirmed, an 11-blade scalpel puncture of the skin should be completed. Drain 1 should then be attached to a 5 cc or 10 cc syringe 14. The device is then depressed carefully through the stab incision and down toward the sheath. The blunt needle 18 should come in contact with the sheath. This will be felt (by the technician). The syringe 14 is back aspirated to ensure no blood. The syringe and needle are disconnected and the connector tubing 15 is placed on the drain Luer Lock 6. This should be done with care so as to not displace the tip of the drain, which should be lying very close to the indwelling sheath. The vacuum reservoir (fluid container) 112 should then be attached to the connector tubing and a vacuum applied.

A butterfly adhesive tape should then be applied to hold drain 1 firmly in place—just as is done for IVs. The outflow tube should be looped and further adhesive tapes applied to the thigh. The loop is the first component that might be disturbed. If the operator is concerned because the drain is short, he or she may consider placing a ligature around the drain and through the skin. If such a suture is placed, it will need to be cut before the drain is removed at some time later, prior to discharge. A covering dressing such as an “Op-Site” may be used over the drain at this time, but not over a retained sheath.

It is common practice to leave the sheath in place until the PTT test has normalized by nature. When the PTT is less than 40 sec., the operator should place normal pressure over the vascular sheath site and pull the sheath, exerting normal techniques used to arrest access sheath bleeding from an artery. The drain may commence collecting blood. Additional pressure should arrest the flow of blood and thus indicate success of the external compression. If a “Fem-Stop” is selected later it should be used in the normal manner.

The patient should then transfer to the floor, and the nursing staff should inspect the vacuum reservoir (fluid container) and the stick site every few minutes for the first hour and on a declining scale after that. At the point the patient is considered ready for discharge, the dressing over the drain should be carefully removed and if a suture is present, it should be cut before removal. A final inspection should be done of the groin to ensure that it is soft and there is no hematoma. If that is so, the drain should be pulled without minimal pressure and a Steri-Strip should be placed over the stab wound and the patient may then be discharged with increasing confidence that his or her vascular access procedure was successful and no complications developed.

It is possible, for example, to combine additional diagnostic features with a percutaneously-inserted hematoma drain. Such features as a micro-manometer could be added to continuously measure and plot the tissue pressure. Provided the patient is resting, tracking the pressure in the tissue would likely increase as a hematoma expanded and pressurized. Such a facility could be useful. Alternatively, it is possible to add an oxygen sensor to this device. As arterial blood is leaked from an artery, the oxygen saturations would show an increasing value. Such a measurement is indirect evidence of continued arterial bleeding. Conversely, if the tissue oxygen saturations fell and stayed low, this would be evidence of non-bleeding. As a further alternative, it is possible to add a pH or [H+] probe to the drain. If a hematoma should occur and then stop enlarging, interstitial pH would drop. Any re-bleeds would likely raise the pH.

For fairly clear reasons, the understanding of the dangerous possibilities for concealed hemorrhage following any surgical procedure has been well understood by surgeons across all disciplines for many years and the medical device industry has provided several different kinds of drains for different requirements. For somewhat obscure reasons, interventionalists are less aware of the dangers of concealed hemorrhage and have relied upon clinical skills to diagnose these when they occur. The femoral artery lies in the groin and is a reasonably accessible site to check for hematomas and false aneurysms. If the groin is noted to be expanding and the patient complaining of remarkable groin pain and a hematoma is found, it is already too late. The hematoma drain of the invention is meant to be an early diagnostic tool to better and more rapidly diagnose failure to control a bleeding blood vessel anywhere in the body. If it occurs, the drain will lessen the development of a major complications by allowing the blood to escape to a visual container and not build up inside the patient's thigh. This will lessen the likelihood of serious secondary complications, including hemodynamic compromise from blood loss, anemia and extensive subcutaneous bruising and pain and possible extensive reparative surgery. By limiting the damage that can develop from such a bleed, the cost to the healthcare system will be substantially reduced.

Other embodiments of the invention will be apparent to those skilled in the art from consideration of the specification and embodiments disclosed herein. Thus, the specification and examples are exemplary only, with the true scope and spirit of the invention set forth in the following claims and legal equivalents thereof.

What is claimed is:

1. A percutaneously-placed body-fluid drain comprising:
a main body including a central lumen, the main body having a distal end and a proximal end;
a distal tip at the distal end of the main body, the distal tip having a low-profile shape for aiding insertion into a patient;
one or more side holes in the distal tip, the side holes being in fluid communication with the central lumen; and
a connector attached to the proximal end of the main body, the connector for connecting the drain to an outflow tube such that the outflow tube is in fluid communication with the central lumen.

2. The drain of claim 1 wherein the lumen includes a non-thrombogenic coating.

3. The drain of claim 1 wherein the distal tip is chamfered to create the low-profile shape.

4. The drain of claim 1 wherein the connector is a Luer Lock.

5. The drain of claim 4 wherein the Luer Lock is a female Luer Lock.

6. The drain of claim 1 wherein the distal tip includes a plurality of side holes, the side holes being arranged in a row in the axial direction of the drain.

7. The drain of claim wherein the central lumen is substantially circular in shape with a radius (r) and the wherein the side holes have a diameter of approximately radius (r).

8. The drain of claim 1 wherein the main body is substantially tubular in shape.

9. The drain of claim 1 further including an outflow tubing and a vacuum reservoir, the outflow tubing connected to the connector of the drain and to the vacuum reservoir such that fluid communication is provided between the vacuum reservoir and the drain.

10. An assembly for percutaneously-inserting a body-fluid drain comprising:
a body-fluid drain comprising:
a main body including a central lumen, the main body having a distal end and a proximal end;
a distal tip at the distal end of the main body, the distal tip having a low-profile shape for aiding insertion into a patient;
one or more side holes in the distal tip, the side holes being in fluid communication with the central lumen; and
a connector attached to the proximal end of the main body, the connector for connecting the drain to an outflow tube such that the outflow tube is in fluid communication with the central lumen;
a needle having a beveled distal end and a syringe connector at a proximal end, wherein the drain is placed co-axially over the needle such that needle goes through the connector and central lumen; and
a syringe connected to the syringe connector of the needle.
